(19) United States
(12) Patent Application Publication
(10) Pub. No.: US 2019/0300459 A1
(43) Pub. Date: Oct. 3, 2019

(54) METHOD FOR IMPROVING THE PRODUCTION OF A CHLORINATED ALKANE BY CHLORINATION OF A CHLOROALKENE USING A DILUENT

(71) Applicant: BLUE CUBE IP LLC, Clayton, MO (US)

(72) Inventors: Max Tirtowidjojo, Clayton, MO (US); Marc Sell, Clayton, MO (US); John D. Myers, Clayton, MO (US)

(21) Appl. No.: 16/373,240

(22) Filed: Apr. 2, 2019

Related U.S. Application Data
(60) Provisional application No. 62/652,064, filed on Apr. 3, 2018.

(51) Int. Cl. C07C 17/04

(52) U.S. Cl. CPC C07C 17/04

(57) ABSTRACT

The present invention provides improved processes for the production of a chlorinated alkane by chlorination of a chloroalkene in the presence of a diluent.

### CROSS REFERENCE TO RELATED APPLICATIONS

The instant application claims the benefit of U.S. Provisional Patent Application Ser. No. 62/652,064, filed Apr. 3, 2018, which is incorporated herein by reference in its entirety.

### FIELD OF THE INVENTION

The present disclosure generally relates to processes for preparing halogenated alkanes.

### BACKGROUND OF THE INVENTION

Halogenated alkanes are useful intermediates for many products including agricultural products, pharmaceuticals, cleaning solvents, solvents, gums, silicones, and refrigerants. The processes to prepare halogenated alkanes are varied and can be time consuming, moderately efficient, and lack reproducibility.

One widely used method for preparing chlorinated alkanes comprises chlorinating an alkene or chloroalkene with a chlorinating agent. On a laboratory scale, these processes have been shown to produce the chlorinated alkane is good yields with moderate selectivity. Yet, on a manufacturing scale, these processes tend to lack reproducibility and require specialized manufacturing equipment to provide adequate temperature control of the process.

One highly sought subset of halogenated alkanes is the chlorinated alkanes. Of these chlorinated alkanes, chlorinated propanes especially 1,1,1,2,3-pentachloropropane (240DB) is in increased demand. 240DB is a useful compound utilized in agriculture products and the new generation refrigerants. U.S. Pat. No. 4,650,914 teaches and discloses a process for preparing 1,1,1,2,3-pentachloropropane. The process comprises dehydrochlorinating 1,1,1,3-tetrachloropropane using an aqueous solution of caustic and a phase transfer catalyst to form a mixture of 1,1,3-trichloropropene and 3,3,3-trichloropropene. 1,1,3-Trichloropropene and 3,3,3-trichloropropene are then chlorinated using chlorine with using UV light. U.S. Pat. No. 4,650,914 does not use a diluent in the chlorination process.

US 2016/0107958 teaches and discloses a continuous process for preparing 1,1,1,2,3-pentachloropropane (240DB) by chlorinating 1,1,3-trichloropropene using chlorine in the presence of UV light wherein the molar ratio of the 240DB to 1,1,3-trichloropropene is less than a 19:1 molar ratio. Since the chlorination reaction is highly exothermic, careful control of the process temperature and addition rate of the chlorine is necessary. If there is a lack of control of the process temperature or the addition rate of the chlorine is too high, the formation of other pentachloropropane isomers, such as 1,1,1,3,3-pentachloropropane, and heavy by-products occurs. On a production scale, it is necessary to control the temperature of the process in order to maintain high selectivity and yield, while minimizing by-product formation.

Developing a manufacturing process for the preparation of halogenated alkanes where the process would be robust, provides higher productivity, a lower level of by-products, a greater overall yield, and lower manufacturing cost would be desirable.

### SUMMARY OF THE INVENTION

In one aspect, disclosed herein are processes for preparing and isolating halogenated alkanes, the process comprising: forming a liquid phase reaction mixture comprising at least one C₂-C₆ haloalkene, a halogenating agent, and a diluent; generating the halogenated alkane; and separating the halogenated from the liquid phase reaction mixture. In one embodiment, the process further comprises utilizing a catalyst, such as an iron containing compound, and UV light.

In one aspect, disclosed herein are processes for preparing and isolating halogenated alkanes via the liquid phase reaction between at least one chloroalkene and a chlorinating agent, wherein the chloroalkene comprises at least three carbon atoms and one chlorine atom. The process comprising: a. forming a liquid phase reaction mixture comprising at least one chloroalkene and at least one diluent; b. adding a chlorinating agent to the liquid phase reaction mixture; c. generating the chlorinated alkane; and d. separating a stream comprising the chlorinated alkane from the liquid phase reaction mixture. The above process may optionally comprise utilizing a catalyst, such as an iron containing compound, and UV light.

In another aspect, disclosed herein are processes for preparing and isolating 1,1,1,2,3-pentachloropropane (240DB) by a liquid phase reaction between 1,1,3-trichloropropene, 3,3,3-trichloropropene, or combinations thereof and a chlorinating agent. The process comprises a. forming a liquid phase reaction mixture comprising 1,1,3-trichloropropene, 3,3,3-trichloropropene, or combinations thereof and at least one diluent; b. adding a chlorinating agent; c. generating the 1,1,1,2,3-pentachloropropane (240DB); and d. separating the 1,1,1,2,3-pentachloropropane (240DB) from the liquid phase reaction mixture.

Other features and iterations of the invention are described in more detail below.

### BRIEF DESCRIPTION OF FIGURES

FIG. 1 is a graphical representation showing the molar ratio of 1,1,1,2,3-pentachloropropane to 1,1,3-trichloropropene versus the % conversion of the process.

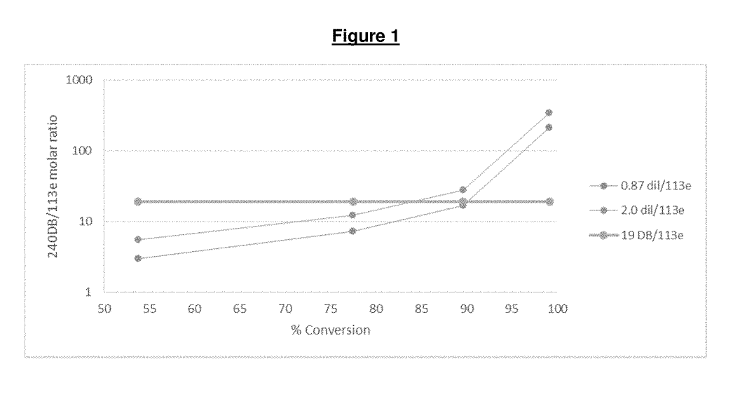

FIG. 2 is a graphical representation showing the % conversion of 1,1,3-trichloropropene and 3,3,3-trichloropropene in the presence of carbon tetrachloride or 1,1,1,2,3-pentachloropropane versus time.

FIG. 3 is a graphical representation of showing the % selectivity of 1,1,1,2,3-pentachloropropane versus % conversion of 1,1,3-trichloropropene and 3,3,3-trichloropropene.

### DETAILED DESCRIPTION OF THE INVENTION

Disclosed herein are processes for the preparation of C₂-C₆ halogenated alkanes. In general, the process comprises preparing a liquid phase reaction by contacting at least one halogenated alkene and a halogenating agent in the presence of at least one diluent. In one embodiment, the halogenated alkane is a chlorinated alkane and the halogenating agent is a chlorinating agent. The halogenated alkene, halogenating agent and the diluent may be combined in any order. Thus, the halogenated alkene may be added to the halogenating agent in the diluent, the three reagents may be combined simultaneously, or the halogenating agent is added to the alkene in the diluent.

In one aspect, disclosed herein are processes for the preparation of chlorinated alkanes. In general, the process comprises preparing a liquid phase reaction by contacting at least one chlorinated alkene in the presence of at least one diluent. The liquid phase is contacted with a chlorinating agent forming a liquid phase reaction mixture thereby generating the chlorinated alkane under conditions detailed below.

The processes have been shown to provide enhanced kinetics, higher productivity, improved temperature control, improved overall yield, and lower overall manufacturing cost.

### (I) Process for the Preparation of Chlorinated Alkanes

One aspect of the present disclosure encompasses processes for the preparation of chlorinated alkanes. The processes comprise forming a liquid phase reaction in a reactor comprising at least one chlorinated alkene and at least one diluent wherein the chloroalkene comprises at least three carbon atoms and one chlorine atom. Once this liquid phase reaction is formed, a chlorinating agent is added forming the liquid phase reaction mixture and the chlorinated alkane is formed. The processes are conducted to a selectivity of at least 50%. After the separation of one or more chlorinated alkenes, light by-products, and the at least one diluent, the chlorinated alkanes are produced in high yield and at a reduced manufacturing cost.

(a) Reaction Mixture

The processes commence by preparing a liquid phase mixture reaction in a reactor comprising at least one chlorinated alkene and at least one diluent. Then the chlorinating agent is added. The chlorinated alkene(s), halogenating agent and the diluent may be combined in any order. Thus, the halogenated alkene may be added to the halogenating agent in the diluent, the three reagents may be combined simultaneously, or the halogenating agent is added to the alkene in the diluent.

(i) Chlorinated Alkene

A wide variety of chlorinated alkenes may be used in the process. As appreciated by the skilled artisan, the at least one chlorinated alkene may be introduced into the reactor as a liquid or a gas. Under conditions of the process as detailed below, the at least one chlorinated alkene may undergo a phase transition where the gas is condensed to a liquid.

Generally, the at least one chlorinated alkene, comprises at least 2 carbon atoms and one chlorine atom. More preferably, the at least one chlorinated alkene, comprises at least 3 carbon atoms and one chlorine atom. In various embodiments, the at least one chlorinated alkene may be linear or cyclic. Additionally, the at least one chlorinated alkene may be wet or dry. In various embodiments, the at least one chlorinated alkene may have a water content below 1000 ppm, i.e. indicating the alkene is essentially dry. In other embodiments, the one or more chlorinated may have a water content above 1000 ppm, i.e. indicating the alkene is wet. Non-limiting examples of linear chlorinated alkenes may be allyl chloride, 2-chloropropene, 3-chloropropene, vinylidene chloride, 1,3-dichloropropene, 2,3-dichloropropene, 3,3-dichloropropene, 1,2,3-trichloropropene, 1,1,3-trichloropropene, 3,3,3-trichloropropene, 1,1,2,3-tetrachloropropene, 2-chloro-1-butene, 3-chloro-1-butene, 2-chloro-2-butene, 1,4-dichloro-2-butene, 3,4-dichloro-1-butene, 1,3-dichloro-2-butene, 2,3,4-trichloro-1-butene, 1,2,3,4-tetrachloro-2-butene, 1,1,2,4-tetrachloro-1-butene, 2,3-dichloro-1,3-butadiene, 1-chloro-3-methyl-2-butene, 3-chloro-3-methyl-butene, 5-chloro-1-pentene, 4-chloro-1-pentene, 3-chloro-1-pentene, 3-chloro-2-pentene, 1,2-dichloro-1-pentene, 1,1,5-trichloro-1-pentene, 6-chloro-1-hexene, 1,2-dichloro-1-hexene, and combinations thereof. Non-limiting examples of cyclic chlorinated alkenes may be chlorocyclopropene, 1-chlorocyclobutene, 1,2-dichlorocyclobutene, 3,4-dichlorocyclobutene, 1-chlorocyclopentene, 2-chlorocyclopentene, 3-chlorocyclopentene, 1,2-dichlorocyclopentene, 4,4-dichlorocyclopentene, 3,4-dichlorocyclopentene, 1-chloro-1,3-cyclopentadiene, 2-chloro-1,3-cyclopentadiene, 5-chloro-1,3-cyclopentadiene, 1,2-dichloro-1,3-cyclopentadiene, 1,3-dichloro-1,3-cyclopentadiene, 1,4-dichloro-1,3-cyclopentadiene, 5,5 dichloro-1,3-cyclopentadiene, 1,2,3-trichloro-1,3-cyclopentadiene, 1,2,3,4-tetrachloro-1,3-cyclopentadiene, 1-chloro-1,3-cyclohexadiene, and combinations thereof.

(ii) Diluent

A large number of diluents may be used in the process. Generally, the at least one diluent may comprise any compound which provides homogeneity to the liquid phase reaction mixture and does not participate and/or is non-reactive in the process. In various embodiments, the at least one diluent may have a water content below 1000 ppm, i.e. indicating the at least one diluent is essentially dry. In other embodiments, the at least one diluent may have a water content above 1000 ppm, i.e. indicating the diluent is wet.

Generally, the at least one diluent comprises a halogenated alkane. In various embodiments, the halogenated alkane is selected from a group consisting of C₁-C₆ chlorinated linear alkanes, C₃-C₆ chlorinated cyclic alkanes, chlorinated arenes, or combinations thereof. Non-limiting examples of C₁-C₆ chlorinated linear alkanes may be 1,2-dichloropropane, 1,1,2-trichloropropane, 1,2,3-trichloropropane, 1,1,1,3-tetrachloropropane, 1,2,2,3-tetrachloropropane, 1,1,1,3,3-pentachloropropane, 1,1,2,3-tetrachloropropane, 1,1,2,2,-tetrachloropropane, 1,1,1,2,3-pentachloropropane, 1,1,1,2,2-pentachloropropane, 1,1,2,2,3-pentachloropropane, hexachloroethane, octachloropropane, decachlorobutane, dodecachloropentane, perchlorohexane, and combinations thereof. Non-limiting examples of C₃-C₆ chlorinated cyclic alkanes may be monochlorocyclopropane, 1,2-dichlorocyclopropane, tetrachlorocyclobutane, hexachlorocyclopropane, octchlorocyclobutane, chlorocyclopentane, trichlorocyclopentane, decachlorocyclopentane, dodecachlorocyclohexane, or combinations thereof. Non-limiting examples of chlorinated arene compounds may be chlorobenzene, chlorotoluene, 1,4-dichlorobenzene, chloronaphthalene, hexachlorobenzene. In another preferred embodiment, the at least one diluent may be carbon tetrachloride (CCl₄), 1,1,1,2,3-pentachloropropane (240DB), or combinations thereof.

The molar ratio of the diluent to the chloroalkene in the stream may be greater than 1. In various embodiments, the ratio of the diluent to the chloroalkene may be greater than 1, greater than 10, greater than 30, greater than 50, greater than 100, or greater than 1000.

The molar ratio of the at least one diluent to the at least one chlorinated alkene may range from 0.000001:1 to about 1000000:1. In various embodiments, the molar ratio of the at least one diluent to the at least one chlorinated alkene may range from 0.000001:1 to about 1000000:1, from 0.00001:1 to about 100000:1, from about 0.0001:1 to about 10000:1, from 0.001:1 to about 1000:1, from about 0.01:1 to about 100:1, or from 0.1 to about 10:1.

(iii) Optional Catalyst

A wide variety of catalysts may be optionally used in the process. In some embodiments, the catalyst may be a transition metal catalyst. As used herein, the term “transition metal catalyst” refers to a transition metal element, a transition metal salt, a transition metal containing alloy, or combinations thereof. Non limiting examples of transition metals in the at least catalytic species may be selected from the group consisting of aluminum, bismuth, chromium, cobalt, copper, gallium, gold, indium, iron, lead, magnesium, manganese, mercury, nickel, platinum, palladium, rhodium, samarium, scandium, silver, titanium, tin, zinc, zirconium, and combinations thereof. In a preferred embodiment, the catalyst may comprise a solid transition metal selected from the group consisting of iron. Non-limiting examples of these preferred catalytic species include iron metal, an iron containing compound, an alloy of iron, an alloy of copper, or combinations thereof.

In some embodiments, the at least one catalyst may comprise a transition metal element, a transition metal alloy, or combinations thereof having an oxidation state of (0). In various embodiments, the iron compound may be part of an alloy with carbon. Non-limiting examples of iron carbon alloys may be alloy of iron and carbon is selected from a group consisting of cast iron, pig iron, wrought iron, anthracite iron, wootz steel, carbon steel, and combinations thereof. For example, the transition metal element, a transition metal alloy, or combinations thereof may be in the form of a foil, a sheet, a screen, a wool, a wire, a ball, a plate, a pipe, a rod, a bar or a powder.

In an embodiment, the at least one catalyst may comprise a transition metal salt. Non-limiting examples of suitable transition metal salts may be an aluminum salt, a bismuth salt, a chromium salt, a cobalt salt, a copper salt, a gallium salt, a gold salt, an indium salt, an iron salt, a lead salt, a magnesium salt, a manganese salt, a mercury salt, a nickel salt, a platinum salt, a palladium salt, a rhodium salt, a samarium salt, a scandium salt, a silver salt, a titanium salt, a tin salt, a zinc salt, a zirconium salt, and combinations thereof. In an embodiment, suitable transition metal salts which may be used in the process may be an iron salt. Non-limiting examples of suitable transition metals salts may be iron (II) salt, iron (III) salt, or combinations thereof. As appreciated by the skilled artisan, a wide variety of anions may be part of the transition metal salt. Non-limiting examples of suitable anions in the transition metal salts may include acetates, acetyacetonates, alkoxides, butyrates, carbonyls, dioxides, halides, hexonates, hydrides, mesylates, octanoates, nitrates, nitrosyl halides, nitrosyl nitrates, sulfates, sulfides, sulfonates, phosphates, and combinations thereof. Non-limiting examples of suitable transition metal salts may include iron (II) chloride, iron (III) chloride, iron (II) bromide, iron (II) iodide, iron (III) bromide, iron (III) oxide, and iron (II, III) oxide.

In various embodiments, the iron catalyst used in the process may be in various oxidation states, such as Fe(0), Fe(II), and Fe(III). In one aspect, the iron catalyst may be Fe(0) alone as elemental iron or an iron carbon alloy. In an additional aspect, the iron catalyst may comprise a mixture of Fe(0) and Fe(II) salt. In another aspect, the iron catalyst may comprise a mixture of Fe(0) and Fe(III) salt. In still another aspect, the iron catalyst may comprise a mixture of Fe(II) salt and Fe(III) salt. In yet another aspect, the iron catalyst may comprise a mixture of Fe(0), Fe(II) salt, and Fe(III) salt.

In various embodiments, the at least one catalyst may be mobilized on the surface of a support. Non-limiting examples of suitable supports may be alumina, silica, silica gel, diatomaceous earth, carbon and clay. Non-limiting examples of iron compounds mobilized on a support may include iron on carbon, iron on diatomaceous earth, and iron on clay.

In another embodiment, the catalyst may be suspended within the reaction mixture or affixed to the reactor below the surface of the reaction mixture so the catalyst contacts the reaction mixture. In other embodiments, the catalyst may be part of a fixed bed or a tray.

As appreciated by the skilled artisan, the catalyst, once in the process, may undergo and oxidation and/or reduction to produce an activated catalytic species in various oxidation states. The oxidation state of these active catalytic species may vary, and may be for examples (I), (II), and (Ill). In one aspect, the active iron catalyst may in the Fe(I) oxidation state. In another aspect, the active iron catalyst may be Fe(II). In still another aspect, the active iron catalyst may be in the Fe(III) oxidation state. In an additional aspect, the active iron catalyst may comprise a mixture of Fe(I) and Fe(II). In still another aspect, the active iron catalyst may comprise a mixture of Fe(I) and Fe(III) oxidation states. In yet another aspect, the active iron catalyst may be in the Fe(II) and Fe(III) oxidation states. In another aspect, the active iron catalyst may in the Fe(I), Fe(II) and Fe(III) oxidation states.

Generally, the surface area of the catalyst may range from 1 cm²/(kg/hr) to about 10,000 cm²/(kg/hr). In various embodiments, the surface area of the metal may range 1 cm²/(kg/hr) to about 10,000 cm²/(kg/hr), from about 100 cm²/(kg/hr) to about 7,500 cm²/(kg/hr), from about 1,000 cm²/(kg/hr) to about 5,000 cm²/(kg/hr), or from 2,000 cm²/(kg/hr) to about 4,000 cm²/(kg/hr).

In general, the weight ratio of the catalyst to the chlorinated alkene may be from 0.0001:1 to about 1000:1. In various embodiments, the weight ratio of the catalyst to the chlorinated alkene may be from 0.0001:1 to about 1000:1, from 0.0001:1 to about 500:1, from about 0.001:1 to about 250:1, from about 0.01:1 to about 100:1, from about 0.1:1 to about 50:1, or from about 1:1 to about 10:1.

(iv) Introduction of the Optional Catalyst(s) into the Process

Generally, the at least one catalyst may be introduced to the process in various ways. In one aspect, the at least one catalyst comprising a metal, a metal alloy, a metal salt(s), or combinations thereof may be introduced directly into the process. In another aspect, a catalyst solution comprising at least one catalyst may be prepared by dissolving at least a portion of the metal, a metal alloy, metal salt(s), or combinations thereof in a mixture of the at least one diluent, the introduced into the reactor. In yet another embodiment, a catalyst solution may be generated inside the reactor by mixing the metal, a metal alloy, metal salt(s), or combinations thereof, the at least one chlorinated alkene, the at least one diluent, and other, optional compounds, such as promoters or ligands. As appreciated by the skilled artisan, other methods for introducing the at least one catalyst or solution of the at least one catalyst into the reactor may be envisioned. The at least one chlorinated alkene may be in the reactor before the catalyst is added, or the at least one chlorinated alkene may be added to the reactor after the catalyst.

(b) Chlorinating Agent

A large number of chlorinating agents may be used in the process. Non-limiting examples of chlorinating agents may be chlorine, sulfuryl chloride, thionyl chloride, oxalyl chloride, phosphorus (Ill) chloride, phosphorus (V) chloride, phosphorus (V) oxychloride, or combinations thereof. In a preferred embodiment, the chlorinating agent is sulfuryl chloride, chlorine gas, or combinations thereof. More preferably, the chlorinating agent is chlorine.

While substoichiometric amounts of chlorinating agents may be used, generally, the chlorinating agent is used in excess. The molar ratio of the chlorinating agent to the chlorinated alkene may range from 1.1:1.0 to about 1000:1. In various embodiments, the molar ratio of the molar ratio of the chlorinating agent to the chlorinated alkene may range from 1.1:1.0 to about 1000:1, from 2.0:1 to about 750:1, from 3.0 to about 100:1, or from 4.0:1 to about 50:1. The chlorinating agent is essentially dry, i.e., it has a water content of the below 1000 ppm. Lower water concentrations are preferred, but not required.

(c) Optional Use of UV Light

In various embodiments, UV light may be used to enhance the reaction. In general, the exposure of UV light to the reaction may occur for a period of a few minutes or throughout the entire process.

(d) Reaction Conditions

As appreciated by the skilled artisan, the above process may be run in a batch mode or a continuous mode, with continuous mode preferred.

In a continuous mode, a stirred tank reactor may be used, or a series of stirred tank reactor to approach the performance of an ideal plug flow reactors may be utilized to improve the overall efficiency of the process. In another embodiment, the process in continuous modes may be stirred in various methods to improve the mixing of the gas-liquid-solid system as appreciated by the skilled artisan.

In general, the process for the preparation of halogenated alkanes will be conducted to maintain the temperature from about 00° C. to about 80° C. using an internal or external heat exchanger. As appreciated by the skilled artisan, the temperature of the reactor is partially maintained by boiling off or vaporizing a portion of the reactants and products. In various embodiments, the temperature of the reaction may be maintained from about 00° C. to about 80° C., from 5° C. to about 50° C., from 10° C. to about 40° C., or from about 15° C. to about 25° C.

Generally, the process may be conducted at a pressure of about atmospheric pressure (˜14.7 psi) to about 200 psi so the amount of the gases and liquid are in suitable quantities so the reaction may proceed and maintain the kinetics of the process. In various embodiments, the pressure of the process may be from about atmospheric pressure (˜14.7 psi) to about 200 psi, from about 20 psi to about 180 psi, from about 40 psi to about 160 psi, from about 80 psi to about 140 psi, or from 100 psi to about 120 psi.

Generally, the reaction is allowed to proceed for a sufficient period of time until the reaction is complete, as determined by any method known to one skilled in the art, such as chromatography (e.g., GC-gas chromatography). The duration of the reaction may range from about 5 minutes to about 6 hours. In some embodiments, the duration of the reaction may range from about 5 minutes to about 6 hours, from about 0.5 hour to about 4 hours, from about 0.75 hours to about 3 hours, or from about 1 hour to about 2 hours.

As appreciated by the skilled artisan, there are many methods to stir the contents of a reactor. These methods would provide increased kinetics of the process while maintaining the temperature of the process. In various embodiments, these methods simply mix the liquid phase of the reaction mixture. Non-limiting methods to adequately stir the liquid phase contents of the reactor may be jet stirring, impellers, baffles in the reactor, or combinations thereof. At least one of these methods may be utilized in the process to maintain the kinetic of the process.

Jet mixing utilizing at least one nozzle, as appreciated by the skilled artisan, withdraws a portion of the reactor effluent and pumps the reactor effluent through at least one nozzle, thereby creating turbulence in the liquid phase. The at least one nozzle may be positioned below the surface of the liquid phase, thereby creating turbulence in the liquid phase and providing increased mixing. The at least one nozzle may be positioned at the surface of the liquid phase or directed through the gas phase (headspace of the reactor) into the liquid phase, thereby providing increased turbulence of the reaction mixture but also provides increased absorption of the gas phase into the liquid phase.

(e) Output from the Process to Produce Halogenated Alkanes

The process, as outlined above, produces halogenated alkanes. In general, the process produces the halogenated alkane in at least 95% selectivity. In various embodiments, the halogenated alkane is produced in a selectivity of at least 95%, in at least 96%, in at least 97%, in at least 98%, in at least 99%, or in at least 99.5%.

In general, the process converts the at least one chlorinated alkene, to the chlorinated alkane in a conversion of at least 50%. The skilled artisan understands the percent conversion (% conversion) is determined by the percentage of the one or more chlorinated alkene, fluorinated alkene, or combinations thereof converted to the chlorinated alkane. In various embodiments, the % conversion of the process is at least 50%, at least 60%, is at least 70%, is at least 80%, is at least 90%, or even at least 99%.

In general, the process produces the chlorinated alkanes in at least 50 weight percent (wt %) in the reaction mixture of the reactor. In various embodiments, the halogenated alkane is produced in at least 50 wt %, in at least 60 wt %, in at least 70 wt %, in at least 80 wt %, in at least 90 wt %, in at least 95 wt %, or in at least 99 wt % in the reaction mixture of the reactor.

In an embodiment, the chlorinated alkane is a pentachloropropane. In a preferred embodiment, the chlorinated alkane is 1,1,1,2,3,-pentachloropropane (240DB).

### (II) Separation of the Halogenated Alkane and Recycle Streams

The first step in the process comprises separating purified chlorinated alkane from the liquid phase reaction mixture of the reactor comprising at least one chlorinated alkene, the chlorinated alkane, chlorinating agent, the at least one diluent, and light by-products by using a separator.

In various embodiments, the separator may be a distillation column or a multistage distillation column which comprises at least one theoretical plate. Non-limiting examples of distillations may be a simple distillation or a vacuum distillation. Additionally, the separator may further comprise a reboiler, a bottom stage, or a combination thereof. Various distillation columns may be used in this capacity. In one embodiment, a side draw column or a distillation column which provides outlet stream from an intermediate stage or a divided wall column (dividing wall column (DWC)) is a single shell, fully thermally coupled distillation column capable of separating mixtures of three or more components into high purity products (product effluent streams) may be used as a separator where the product effluent streams comprise chlorinated alkene, the chlorinated alkane, chlorinating agent, heavy by-products, light by-products, or combinations thereof. A portion of the product effluent streams produced by the process may be recycled back into the reactor to provide increased kinetics, increased efficiencies, reduced overall cost of the process, increased selectivity of the desired chlorinated alkane, and increased yield of the desired chlorinated alkane.

As appreciated by the skilled artisan, separating the purified chlorinated alkane from the liquid phase reaction mixture of the reactor would produce at least two product effluent streams. In various embodiments, separating the purified chlorinated alkane may produce three, four, or more product effluent streams depending on the separation device utilized. As an example, the separation of the chlorinated alkane from the contents of the liquid phase reaction mixture using two product effluent streams is described below.

The process utilizing one separator commences by transferring at least a portion of the liquid phase reaction mixture (also termed reactor effluent) from the reactor to the separator. In this operation, all or some of the reactor effluent may be separated into two distinct product effluent streams, product effluent streams (a) and (b). Product effluent stream (a) comprises light by-products, at least one chlorinated alkene, and at least one diluent which is separated from product effluent stream (b) which comprises the chlorinated alkane and any heavies. Product effluent stream (a) comprising light by-products, the at least one chlorinated alkene, and the at least one diluent. If desired, product effluent stream (a) may be transferred to a second separator, where two additional product effluent streams (c) and (d) are produced. Product effluent stream (c) comprises the light by-products and at least one chlorinated alkene while product effluent stream (d) comprises the at least one diluent. Product effluent stream (c) may be further transferred into additional separators to achieve the desired purity of the at least one chlorinated alkene. Product effluent stream (b) comprising the chlorinated alkane may be transferred into additional separators to achieve the desired purity of the chlorinated alkane. Any resulting heavies may be recycled to the reactor, sent to a different process, discarded or combinations thereof.

In various embodiments, at least a portion of product effluent streams (a), (c), and/or (d) may be recycled back into the reactor zone. These product effluent streams may also be fed into another process to produce other products. These steps may be performed in order to improve the efficiency, reduce the cost, reduce contaminants, reduce waste, and increase through-put of the process.

In another embodiment, at least a portion of product effluent streams (a), (c) and/or (e) may be mixed with fresh liquid feed before being recycled back into the reactor zone in batch mode or continuous mode. In various embodiments, the recycle product effluent streams and fresh liquid feed streams may be introduced into the reactor separately or mixed together before entering the process. These product effluent streams and fresh liquid feed streams comprise the chlorinated alkene and the diluent. The introduction of these fresh liquid feeds into the reactor or mixing the recycle product effluent streams with fresh feeds increases the efficiency of the process, reduces the overall cost, maintains the kinetics, increase the through-put, and reduces the by-products produced by the process. The amounts of the product effluent streams recycled and fresh liquid feed streams added to the reactor may be the same or different. One way to measure the amount product effluent streams recycled and fresh liquid feed streams being added to the reactor is to identify the mass flow of each of these streams. The product effluent streams being recycled to the reactor has a product effluent streams mass flow, while the fresh liquid feeds being added to the reactor has a fresh liquid feed mass flow. Mass flows may be measured using methods known in the art.

Generally, the mass ratio of the product effluent stream mass flow being recycled to the fresh material feed mass flow is adjusted to not only maintain the conversion of the process but also maintain the kinetics of the process.

Product stream (b) comprising the chlorinated alkane produced in the process may have a yield of at least about 20%. In various embodiments, product stream (b) comprising the chlorinated alkane produced in the process may have a yield of at least about 30%, at least about 50%, at least about 70%, at least about 75%, at least about 80%, at least about 85%, at least about 90%, at least about 95%, or at least about 99%.

Product stream (b) comprising the chlorinated alkane produced in the above process may have a weight % of at least 95 wt %. In various embodiments, the weight percentage of the chlorinated alkane may have a weight percentage of at least 95 wt %, of at least 96 wt %, of at least 97 wt %, of at least 98 wt %, of at least 99 wt %, or at least 99.5 wt %.

### (III) Preferred Embodiments: 1,1,1,2,3-Pentachloropropane

(a) Process for the Preparation of 1,1,1,2,3-Pentachloropropane

Another aspect of the present disclosure encompasses processes for the preparation of 1,1,1,2,3-tetrachloropropane (240DB). The process commences by preparing a liquid phase comprising 1,1,3-trichloropropene, 3,3,3-trichloropropene, or combinations thereof, and at least one diluent. This liquid phase is contacted with the chlorinating agent producing 1,1,1,2,3-pentachlorpropane. The at least one diluent is described above in Section (I)(a)(ii). Suitable optional catalysts are described in Section (I)(a)(iii). The chlorinating agent is described above in Section (I)(b) In a preferred embodiment, the at least one chlorinated alkene comprises 1,1,3-trichloropropene, 3,3,3-trichloropropene, or combinations thereof and chlorinating agent is chlorine. In a preferred embodiment, the at least one diluent is carbon tetrachloride.

(b) Reaction Conditions

The reaction conditions are described above in Section (I)(d).

(c) Output from the Process to Prepare 1,1,1,2,3-Pentachloropropane

In a preferred embodiment, the process produces 1,1,1,2,3-pentachloropropane. As appreciated by the skilled artisan, the process is conducted to minimize the formation of byproducts and maximize the formation of 1,1,1,2,3-pentachloropropane by maximizing the selectivity of 1,1,1,2,3-pentachloropropane. After removal of light-by-products, the at least one chlorinated alkene, and the at least one diluent, the 1,1,1,2,3-pentachloropropane may be used directly without further purification or the 1,1,1,2,3-pentachloropropane may be further purified to achieve the desired purity.

The process, as outlined above, produces 1,1,1,2,3-pentachloropropane. In general, the process produces the 1,1,1,2,3-pentachloropropane in at least 95% selectivity. In various embodiments, the 1,1,1,2,3-pentachloropropane is produced in a selectivity of at least 95%, in at least 96%, in at least 97%, in at least 98%, in at least 99%, or in at least 99.5%.

In general, the process converts 1,1,3-trichloropropene, 3,3,3-trichloropropene, or combinations thereof, to 1,1,1,2,3-pentachloropropane in a conversion of at least 50%. The skilled artisan understands the percent conversion (% conversion) is determined by the percentage of 1,1,3-trichloropropene, 3,3,3-trichloropropene, or combinations thereof converted to 1,1,1,2,3-pentachloropropane. In various embodiments, the % conversion of the process is at least 50%, at least 60%, is at least 70%, is at least 80%, is at least 90%, or even at least 99%

In general, the process produces the 1,1,1,2,3-pentachloropropane in at least 20 weight percent (wt %) in the reaction mixture of the reactor. In various embodiments, the 1,1,1,2,3-pentachloropropane is produced in at least 20 wt %, in at least 50 wt %, in at least 70 wt %, in at least 80 wt %, in at least 90 wt %, in at least 95 wt %, or in at least 99 wt % in the reaction mixture of the reactor.

(d) Separation of 1,1,1,2,3-Pentachloropropane and Recycle Streams

The separation of the 1,1,1,2,3-pentachloropropane and recycle streams is described in Section (II).

1,1,1,2,3-Pentachloropropane produced in the process may have a yield of at least about 20%. In various embodiments, the 1,1,1,2,3-pentachloropropane produced in the process may have a yield of at least about 20%, at least about 50%, at least about 70%, at least about 75%, at least about 80%, at least about 85%, at least about 90%, at least about 95%, or at least about 99%.

1,1,1,2,3-Pentachloropropane produced in the above process may have a weight % of at least 95 wt %. In various embodiments, the weight percentage of the 1,1,1,2,3-pentachloropropane may have a weight percentage of at least 95 wt %, of at least 96 wt %, of at least 97 wt %, of at least 98 wt %, of at least 99 wt %, or at least 99.5 wt %.

### (IV) Further Reaction of the Halogenated Alkanes

In one aspect, disclosed herein are processes for the conversion of chlorinated alkanes, such as 1,1,1,2,3-pentachloropropane, to one or more hydrofluoroolefins. These processes comprise contacting the chlorinated alkanes with a fluorinating agent in the presence of a fluorination catalyst, in a single reaction or two or more reactions. These processes can be conducted in either gas phase or liquid phase with the gas phase being preferred at temperatures ranging from 50° C. to 400° C.

Generally, a wide variety of fluorinating agents can be used. Non-limiting examples of fluorinating agents include HF, F₂, CIF, AlF₃, KF, NaF, SbF₃, SbF₅, SF₄, or combinations thereof. The skilled artisan can readily determine the appropriate fluorination agent and catalyst. Examples of hydrofluoroolefins that may be produced utilizing these processes include, but are not limited to 2,3,3,3-tetrafluoroprop-1-ene (HFO-1234yf), 1,3,3,3-tetrafluoroprop-1-ene (HFO-1234ze), 3,3,3-trifluoroprop-1-ene (HFO-1243zf), and 1-chloro-3,3,3-trifluoroprop-1-ene (HFCO-1233zd).

### Definitions

When introducing elements of the embodiments described herein, the articles “a”, “an”, “the” and “said” are intended to mean that there are one or more of the elements. The terms “comprising”, “including” and “having” are intended to be inclusive and mean that there may be additional elements other than the listed elements.

The term “Tet” refers to carbon tetrachloride.

The term “240DB” refers to 1,1,1,2,3-pentachloropropane.

The term “113e” refers to 1,1,3-trichloropropene.

The term “333e” refers to 3,3,3-trichloropropene.

Having described the invention in detail, it will be apparent that modifications and variations are possible without departing from the scope of the invention defined in the appended claims.

### EXAMPLES

The following examples illustrate various embodiments of the invention.

### Example 1: Preparation of 1,1,1,2,3-Pentachloropropane (240DB)

A mixture comprising 7.4 g of 1,1,3-trichloropropene and 6.84 g of CCl₄ was reacted with Cl₂ in a stirred reactor at atmospheric pressure and 15° C. About 7.2 gr of Cl₂ was sparged into the reactor at a stirring rate of 300 rpm for 1.13 hr.

As can be seen in Table 1, about 99% conversion of 113e was obtained after 1.1 hr with higher than 97% selectivity.

TABLE 1

| | 1,1,3-Trichloropropene | |
|---|---|---|
| Time (Hours) | Conversion | 240 DB % Selectivity |
| 0.2 | 53.8 | 97.4 |
| 0.5 | 77.5 | 97.6 |
| 0.8 | 89.7 | 97.6 |
| 1.133 | 99.2 | 97.2 |

### Example 2: Preparation of 1,1,1,2,3-Pentachloropropane (240DB) without Diluent

13.96 g of 1,1,3-trichloropropene was reacted with Cl₂ in a stirred reactor at atmospheric pressure and 15° C. About 9.4 gr of Cl₂ was sparged into the reactor at a stirring rate of 300 rpm for 1.25 hr.

As can be seen from Table 2, the pure 1,1,3-trichloropropene chlorination without using diluent showed slower kinetics while maintaining the same or slightly lower selectivity.

TABLE 2

| | 1,1,3-Trichloropropene | |
|---|---|---|
| Time (Hours) | Conversion | 240 DB % Selectivity |
| 0.25 | 29.8 | 97.1 |
| 0.583 | 70.0 | 97.6 |
| 1.25 | 98.2 | 97.3 |

### Example 3: Chlorination Rate of 240DV v 113e or 333e

About 21.6 g of crude 1,1,1,2,3-pentchloropropane (240DB) (prepared from the chlorination of 1,1,3-trichloropropene and 3,3,3-trichloropropene) was reacted with Cl₂ at 80° C. for 315 min with as stirring rate of 400 RPM. Upon completion of the reaction of 113e and 333e with Cl₂ (i.e. 100% conversion), the amount of the heavies increased from 6.12 wt % to 7.87 wt %. This result shows the chlorination rate of 240DB to produce heavier byproduct is more than 500× slower than the chlorination rate of 113e or 333e.

### Example 4: Preparation of 1,1,1,2,3-Pentachloropropane (240DB) with Diluent

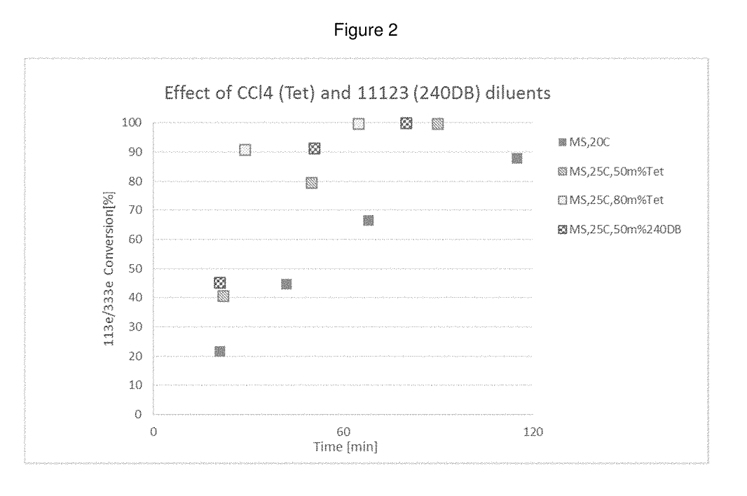

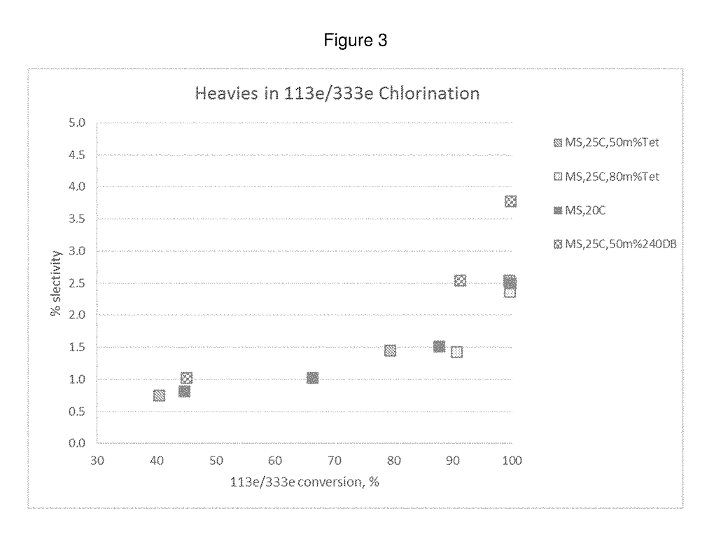

About 27.5 g of a mixture of 113e and 333e was reacted with Cl₂ at a temperature between 20 C and 25 C with a stirring rate of 400 rpm. Cl₂ was added at a rate of 0.2 g/min. As shown in FIG. 2, adding Tet (carbon tetrachloride) as a diluent by 50 mole % and 80 mole % increases the conversion at 20 min residence time by double and more than triple. These experiments show that increasing amount of diluent surprisingly improves the reaction kinetics despite of the lower trichloropropene concentration. FIG. 2 also shows that replacing Tet with 240DB also improves the kinetics as described above. The production of heavy byproduct with boiling point higher than 240DB shown in FIG. 3 indicates that the impact of adding the at least one diluent has little impact on the product selectivity.

What is claimed is:

1. A process for producing a chlorinated alkane, the process comprising:
a. preparing a liquid phase reaction mixture comprising at least one chloroalkene, and at least one diluent;
b. adding a chlorinating agent to the liquid phase reaction mixture;
c. generating a chlorinated alkane; and
d. separating a stream comprising the chlorinated alkane and at least some of the chlorinated alkene from the liquid phase reaction mixture;
wherein the at least one chloroalkene comprises at least three carbon atom and one chlorine atom;
wherein the at least one diluent comprises a halogenated alkane.

2. The process of claim 1, wherein the chloroalkene is 1,1,3-trichloropropene; 3,3,3-trichloropropene; or combinations thereof; and the chlorinating agent comprises chlorine, sulfuryl chloride, thionyl chloride, phosphorus (111) chloride, phosphorus (V) chloride, or combinations thereof.

3. The process of claim 1, wherein the at least one diluent comprising a halogenated alkane is selected from the group consisting of C₁-C₆ chlorinated alkane, chlorinated arene, and combinations thereof.

4. The process of claim 3, wherein the at least one diluent has a lower boiling point than the chlorinated alkane product.

5. The process of claim 3, wherein the at least one diluent is carbon tetrachloride (CCl₄), a chlorinated alkane, or combinations thereof.

6. The process of claim 3, wherein at least a portion of the at least one diluent is recycled back to the reaction zone.

7. The process of claim 3, wherein the at least one diluent comprises the chlorinated alkane product.

8. The process of claim 1, wherein the chlorinated alkane is selected from a group comprising carbon 1,2-dichloropropane; 1,1,2-trichloropropane; 1,2,3-trichloropropane; 1,1,1,3-tetrachloropropane; 1,2,2,3-tetrachloropropane; 1,1,1,3,3-pentachloropropane; 1,1,2,3-tetrachloropropane; 1,1,2,2,-tetrachloropropane; 1,1,1,2,3-pentachloropropane; 1,1,1,2,2-pentachloropropane; 1,1,2,2,3-pentachloropropane; and combinations thereof.

9. The process of claim 1, wherein the chlorinated alkane comprises 1,1,1,2,3-pentachloropropane.

10. The process of claim 1, wherein the molar ratio of the chlorinated alkane to the chloroalkene in the stream is greater than 1.

11. The process of claim 1, wherein the molar ratio of the at least one diluent to the chloroalkene in the liquid phase reaction mixture may range from 0.000001 to 1 to about 1000000 to 1.

12. The process of claim 1, wherein the process further comprises a catalyst.

13. The process of claim 12, wherein the catalyst comprises at least one iron containing compound.

14. The process of claim 13, wherein the iron compound is selected from the group consisting of Fe metal, Fe(II) chloride, Fe(III) chloride, and combinations thereof.

15. The process of claim 1, wherein the process further comprises exposing the liquid phase reaction mixture to UV light.

16. The process of claim 1, wherein the selectivity of the process is at least 50%.

17. A process for producing 1,1,1,2,3-pentachloropropane, the process comprises:
a. preparing a liquid phase reaction mixture comprising 1,1,3-trichloropropene 3,3,3-trichloropropene, or combinations thereof, and at least one diluent;
b. adding a chlorinating agent comprising Cl₂ to the liquid phase reaction mixture;
c. generating 1,1,1,2,3-pentachloropropane; and
d. separating a product effluent stream comprising the 1,1,1,2,3-pentachloropropane from the liquid phase reaction mixture;
wherein the at least one diluent comprises CCl₄; 1,1,1,2,3-pentachloropropane;
or combinations thereof.

18. The process of claim 17, wherein a portion of the product effluent stream comprising the diluent, 1,1,3-trichloropropene, 3,3,3-trichloropropene, or combinations thereof, the chlorinating agent, or combinations thereof is recycled back to the reactor.

19. The process of claim 17, wherein fresh material feeds comprising 1,1,3-trichloropropene 3,3,3-trichloropropene, or combinations thereof, and at least one diluent, a chlorinating agent, or combinations thereof are introduced into the liquid phase reaction mixture.

20. The process of claim 18, wherein the material being recycled to the reactor has a product effluent stream mass flow, while the fresh material feeds comprising 1,1,3-trichloropropene 3,3,3-trichloropropene, or combinations thereof, and at least one diluent, a chlorinating agent, or combinations thereof, that are introduced into the reactor have a fresh material feed mass flow, wherein the mass ratio of the product effluent stream mass flow to the fresh material feed mass flow is adjusted to maintain the conversion of the process and to maintain the kinetics of the process.
